(19) United States
(12) Patent Application Publication
(10) Pub. No.: US 2025/0375191 A1
(43) Pub. Date: Dec. 11, 2025

(54) URINALYSIS SAMPLE COLLECTION AND TESTING DEVICE

(71) Applicant: Jacqueline Aquino, Upper Holland, PA (US)

(72) Inventor: Jacqueline Aquino, Upper Holland, PA (US)

(21) Appl. No.: 19/230,663

(22) Filed: Jun. 6, 2025

Related U.S. Application Data
(60) Provisional application No. 63/657,052, filed on Jun. 6, 2024.

(51) Int. Cl. A61B 10/00

(52) U.S. Cl. CPC A61B 10/007

(57) ABSTRACT

An integrated urine collection and testing device is provided. The device has a folded, sealed configuration in which sample test strips are enclosed in an interior volume defined by an inner surface of the device's substrate and an inner surface of a removable longitudinal sealing section that connects the two sides. Each side has a wing, and when the device is in a collection configuration, the wings are foldable over one another to encircle one end of the substrate while leaving an opposite end of the substrate open. When folded over one another, the wings define an anatomical alignment ledge that generally conforms to the outline of a woman's mons pubis, allowing the woman to hold the wings in alignment therewith so that the device is oriented with the sample test strip end vertically lower than the wings. As a result, during a urination event, the urine impinges the inner substrate surface above the sample test strips and flows over them via gravity. The structure provides for simpler and more hygienic at-home urine collection and testing.

### CROSS-REFERENCE TO RELATED APPLICATIONS

This application claims the benefit of U.S. Provisional Patent Application No. 63/657,052, filed on Jun. 6, 2024, the entirety of which is hereby incorporated by reference.

### FIELD

The present disclosure concerns devices for collecting and testing urine, and more specifically, devices that integrally incorporate laminar flow testing strips and which at the same time can be manually reconfigured from a folded sealed configuration to an inverted and open-ended funnel collection configuration and to a flat testing configuration.

### BACKGROUND

Urine analysis is a common practice for health monitoring at home, including pregnancy tests and general wellness checks, as well as in clinical settings for diagnosing conditions such as urinary tract infections, kidney diseases, and metabolic disorders. Traditional urine collection methods often involve standard urine cups, which present significant challenges, particularly for females. Anatomical differences can lead to difficulties in accurate sample collection, resulting in spillage and contamination. These issues compromise the reliability of test results and cause discomfort and embarrassment for users. Additionally, the positioning required for using standard urine cups is often awkward and inconvenient, especially for elderly women, pregnant women, and those with physical disabilities. These limitations are further pronounced in outdoor activities such as camping, where traditional facilities may not be available. Thus, a device is needed that addresses these challenges by providing a more user-friendly, hygienic, and accurate solution for urine collection, designed to meet the diverse needs of female users in various settings, from home use to clinical environments and beyond.

### SUMMARY

In accordance with a first aspect of the present disclosure, a urinalysis and collection device is provided. The device has a sealed configuration, a collection configuration, and a testing configuration. The device comprises substrate having an outer substrate surface and an inner substrate surface, and a sample testing section comprising at least one sample test strip disposed on the inner substrate surface. In the testing configuration, a first portion of the at least one sample test strip is covered, and a second portion of the sample test strip is uncovered, and in the sealed configuration, the substrate is folded and entirely encloses the at least one sample test strip. In certain examples, two wings are provided at one end of the substrate which are oppositely positioned one another and which can be overlapped to create a closed end during a urine collection event, and the wings define an anatomical alignment edge that allows the device to be consistently and reliably positioned on the female anatomy to receive urine and cause it to contact the at least one sample test strip.

In accordance with a second aspect of the present disclosure, a method of collecting and testing female urine is provided. The method comprises providing a urinalysis collection and testing device comprising a substrate having an inner substrate surface and an outer substrate surface, a first end spaced apart from a second end along a length axis, and first and second substrate sides adjacent one another along a width axis, wherein the first substrate side has a first wing, the second substrate side has a second wing parallel to the first wing. The method further comprises folding the first and second wings toward one another to overlap the first and second wings, thereby covering a proximal portion of the inner substrate surface and defining an anatomical alignment ledge for aligning the device with an anatomical structure. The method also comprises directing urine onto a distal portion of the inner surface of the substrate between the anatomical alignment ledge and the second end of the substrate along the length axis.

### BRIEF DESCRIPTION OF THE FIGURES

The embodiments are illustrated by way of example and not limitation in the accompanying drawings, in which like references indicate similar elements, and in which:

FIG. 1 is a side elevational view of a urinalysis collection and testing device in accordance with a first aspect of the present disclosure, showing the device in a sealed condition.

FIG. 2 is a top plan view of the urinalysis collection and testing device of FIG. 1 shown in a testing configuration;

FIG. 3 is a side elevational view of the urinalysis collection and testing device of FIG. 1 showing a longitudinal sealing section just following its removal from the device's substrate;

FIG. 4 is a perspective view of the urinalysis collection and testing device of FIG. 1 shown in the process of being placed in the collection configuration;

FIG. 5 is a perspective view of the urinalysis collection and testing device of FIG. 1 in a collection configuration;

FIG. 6 depicts a woman in a seated position using the urinalysis collection and testing device of FIG. 1 in a collection configuration to collect and test urine;

FIG. 7 is a side-elevational view of the urinalysis collection and testing device of FIG. 1 with the wings folded to partially overlap one another during the removal of a width-wise sealing section as the device is converted from a collection configuration to a testing configuration;

FIG. 8 is perspective view of the urinalysis and collection device of FIG. 1 just prior to its placement in a testing configuration;

FIG. 9 is a perspective view of the urinalysis collection and testing device of FIG. 1 being imaged by a smartphone to read the test strips; and

FIG. 10 is a top plan view of a modified version of the urinalysis and collection device of FIG. 1 in a testing configuration.

### DETAILED DESCRIPTION

In certain preferred examples, the urinalysis collection and testing devices described herein provide an integrated urine collection and testing device having a sealed configuration in which sample testing strips are supplied with and sealed within the device. The sample testing strips are also attached and located within the natural urine flow path that exists when the device is in a collection configuration such that the results of the test can be readily and reliably viewed without any manipulation of the strips. These features streamline the testing process, reducing steps and time required while simplifying procedures. As a result, the system enhances hygiene and minimizes mess, offering a user-friendly experience for efficient and hassle-free urine analysis.

As explained in detail herein, in preferred examples, the user can readily convert the urinalysis and collection device from its sealed configuration to its collection configuration by detaching a longitudinal, removable sealing section that connects two sides of the device. The sides of the device are foldable about a longitudinal midline. In the collection configuration, the device has the general shape and appearance of a closed-end scoop or an inverted, partial funnel in which the inner surface is exposed along a distal portion its length. In addition, in the collection configuration, two “wings” proximate the closed end of the device define an anatomical alignment ledge that allows the user to position the device in an anatomical location that will allow urine to be reliably received and transported to the sample testing strips. The ‘anatomical alignment ledge’ facilitates easy adjustment of the funnel's position, allowing users to effortlessly align it against their mons pubis, even during midstream urine collection. This feature ensures precise alignment for accurate testing results. The ‘inverted funnel ramp mechanism’ provided in the collection configuration functions as the ergonomic device, providing a comfortable grip for users. Additionally, it houses the test strips, guides the flow of urine onto the strips, and ensures minimal splashing during the collection process.

The device can be readily converted from the collection configuration to a testing configuration by removing a width-wise sealing section and folding the sides about the substrate's longitudinal midline until the inner substrate surfaces are coplanar, which is particularly well-suited for lateral flow assay/laminar flow sample test strips that rely on capillary action to transport the urine in laminar flow to the test strip's reagent. Examples of such laminar flow test strips include commercially available test strips for hormone tests like LH ovulation strips, pregnancy tests for hCG, and drug tests,

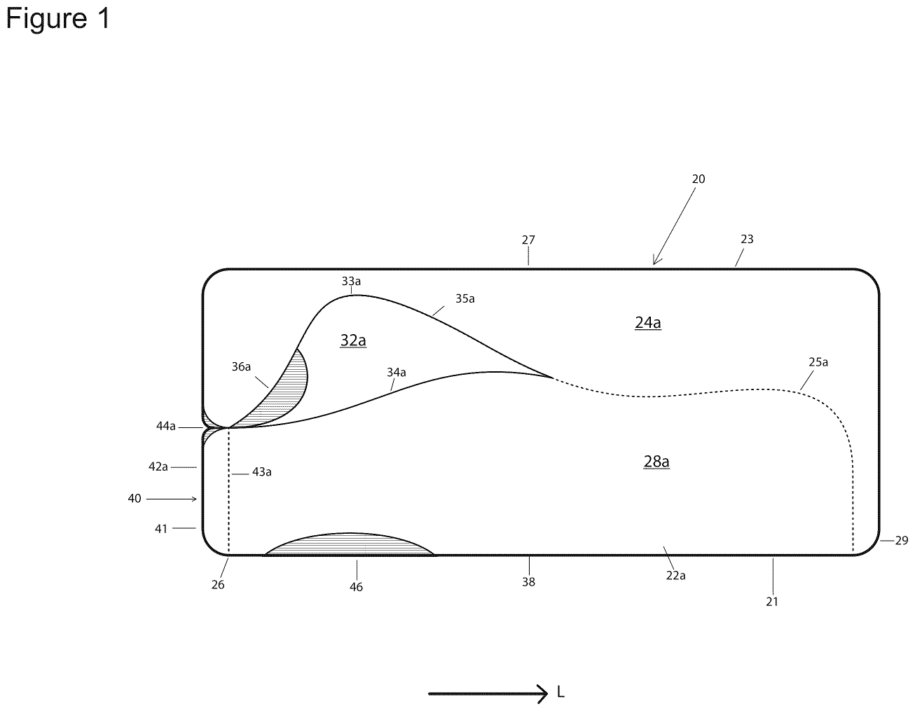

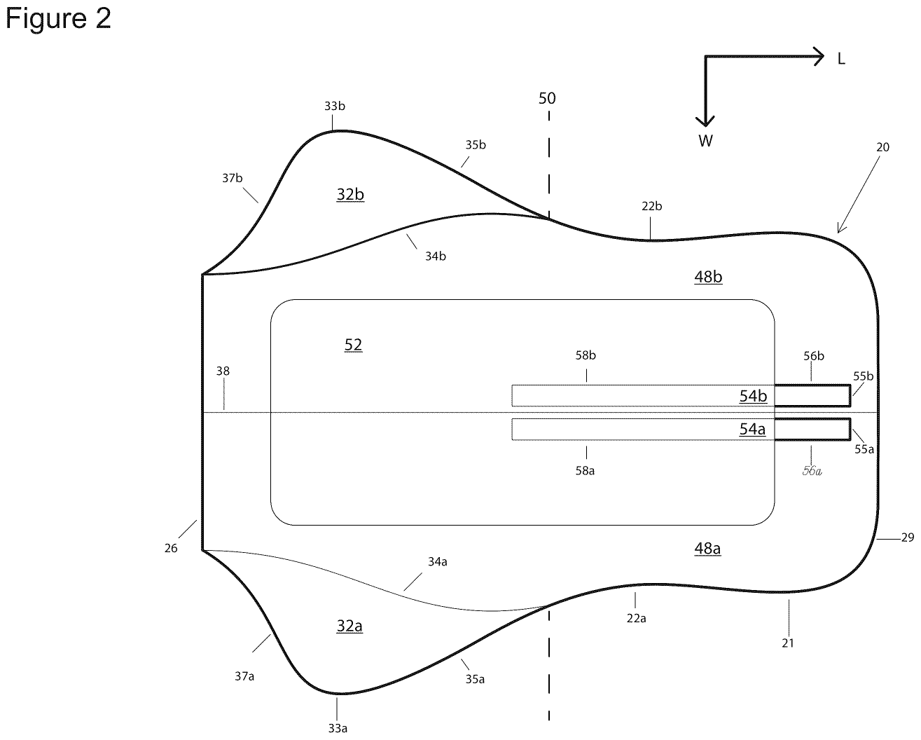

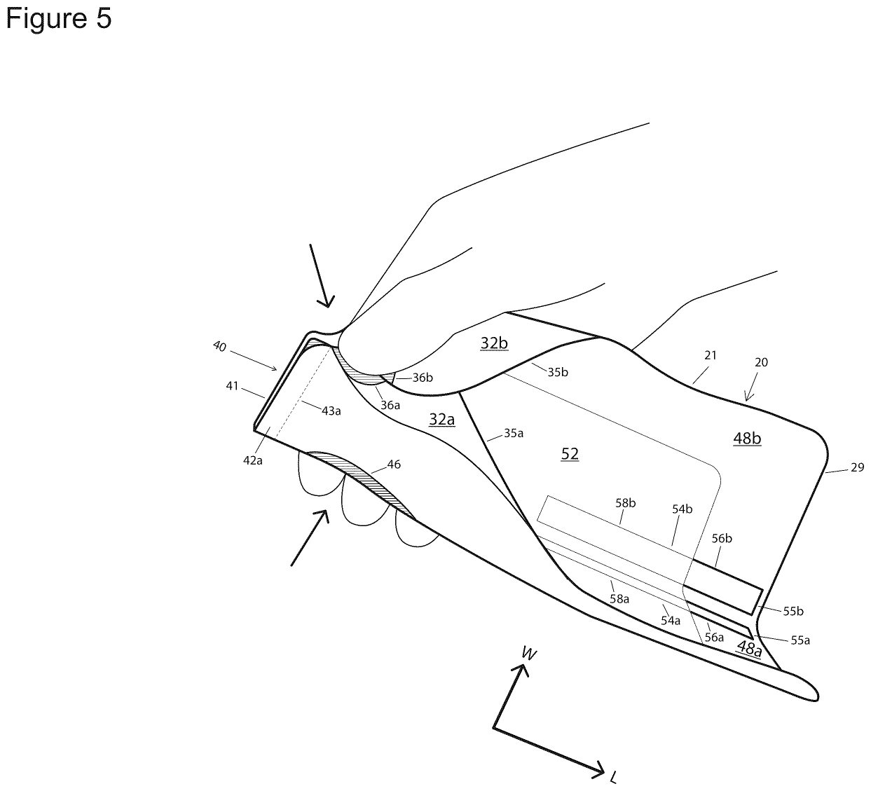

Referring to FIGS. 1-2, a urinalysis and collection device 20 is depicted. Device 20 comprises substrate 21, sample testing strips 54a, 54b, and sample covering layer 52. In FIG. 1 device 20 is shown in a sealed configuration in which sample test strips 54a and 54b (not visible in FIG. 1) are fully enclosed within substrate 21. Device 20 can be placed into three configurations: a sealed configuration in which the sealing strips 54a, 54b are fully enclosed within substrate 21 (FIG. 1), a collection configuration in which device 20 has the shape of a closed-end scoop or an inverted funnel with an open end section at which sample test strips 54a and 54b are exposed (FIG. 5), and a testing configuration in which the substrate 21 is substantially flat, which in turn allows sample test strips 54a and 54b to lay flat, as is preferred in the when sample test strips 54a and 54b are lateral flow assays in which capillary action moves fluid horizontally in laminar flow. As described further below, these configurations beneficially minimize or eliminate the user's loading of test strips into a device, urinating into a cup, and urine to skin contact.

Sample test strips 54a and 54b include those that are commercially available for hormone tests like LH ovulation strips, pregnancy tests for hCG, drug tests, and more. Sample strips 54a and 54b may be used for the same or different tests.

Substrate 21 preferably has the structural integrity and flexibility to be selectively deformable by the user to reconfigure device 20 from the sealed configuration of FIG. 1 into the collection configuration of FIG. 5 and then into the testing configuration of FIGS. 2, 9, and 10. Substate 21 is preferably formed from a paperboard, and more preferably one that resists the incursion of water into the interior of device 20, including from atmospheric moisture. In one example, a recycled paperboard with a waterproof coating is used. Recycled paperboard provides structural integrity, while waterproof coatings ensure the material remains durable and hygienic during use. This material is particularly suitable due to its sustainability and strength. In certain examples, a desiccant pack is enclosed within the interior of device 20 in the sealed configuration.

Another suitable material for substrate 21 is synthetic polypropylene paper, which is a synthetic paper made from polypropylene, known for its durability, water resistance, and tear resistance. It is particularly beneficial for parts of the device 20 that require water resistance and chemical inertness. It offers excellent printability for instructional graphics and user guidelines, and it does not impede laminar flow, ensuring that the sample test strips 54a and 54b remain uncontaminated and easy to read. One suitable example of commercially available polypropylene paper is YUPO® synthetic paper supplied by the Yupo Corporation of Chesapeake, Virginia.

In the sealed configuration, device 20 comprises substrate 21, longitudinal scaling section 23 and width-wise scaling section 40. In FIG. 2, the device 20 is in a testing configuration in which the longitudinal sealing section 23 and width-wise sealing section 40 have been removed. As FIGS. 1 and 2 indicate, substrate 21 comprises first and second sides 22a and 22b which are separated by and foldable about a longitudinal midline 38. The longitudinal midline 38 may be scored, thinned, or otherwise weakened to allow first and second sides 22a and 22b to be folded toward and away from one another about the longitudinal midline 38. Sides 22a and 22b have corresponding first and second side inner substrate surfaces 48a and 48b and outer surfaces 28a and 28b. Thus, in the sealed configuration of FIG. 1, the side inner substrate surfaces 48a and 48b are in facing opposition to one another. However, in the testing configuration of FIGS. 2, first and second side inner substrate surfaces 48a and 48b face in the same direction and are coplanar and parallel to one another. Longitudinal sealing section 23 may have a sealed border at which sides 24a and 24b are adhesively sealed to prevent moisture. air or contaminant incursions at the first and second substrate ends 26, 29. Thus, in preferred examples, the sample test strips 54a, 54b provided in a sterile, pre-loaded condition requiring little or no user intervention.

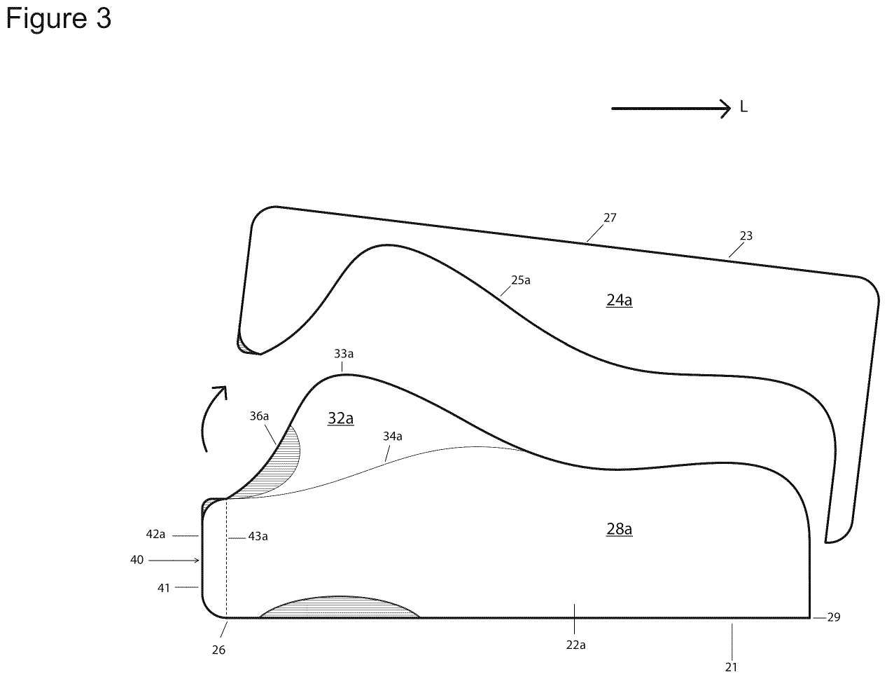

In the sealed configuration of FIG. 1, longitudinal sealing section 23 is attached to each of sides 22a and 22b along respective perforations 25a and 25b (not visible in FIG. 1). Longitudinal sealing section 23 also has a longitudinal mid-line 27 about which sides 24a and 24b are folded in the sealed condition of FIG. 1. Notches 44a and 44b allow the user to readily initiate separation along perforations 25a and 25b to separate each side 24a and 24b of the longitudinal sealing section 23 from its respective substrate side 22a and 22b, as best seen in FIG. 3.

In addition, width-wise sealing section 40 has two sides 42a and 42b joined together at longitudinal midline (“longitudinal” referring to the length of the width-wise sealing section 40, not the length of substrate 21 or its length axis L). Perforations 43a and 43b allow the width-wise sealing section 40 to be detached from each of sides 22a and 22b of substrate 21. The term “width-wise” is used because the width-wise sealing enclosure prevents sides 22a and 22b from separating in the width (“W”) direction (FIG. 2) and thus first end 26 of substrate 21 is closed in the collection configuration, i.e., urine cannot exit first end 26. In FIG. 1 the width axis W is normal to the page and defined by the testing configuration of FIG. 2.

As best seen in FIG. 2, in the testing configuration of FIG. 2, wings 32a and 32b project away from the longitudinal axis 38 and from one another along the width axis W. Each wing 32a and 32b has a respective maximum point 33a and 33b, which defines the farthest point on the wing relative to the longitudinal midline 38 along the width axis W. Each wing 32a and 32b has a curved shape with a single maximum point 33a, 33b. A variety of different wing 32a, 32b shapes may be used. However, the wing 32a, 32b geometry is preferably selected to ensure that the wings 32a and 32b are capable of overlapping along the width axis W when the width-wise sealing section 40 is attached so that the wings 32a and 32b can collectively act as a splash guard so that wing sides 35a and 35b can cooperatively define an anatomical alignment ledge 37 (See FIG. 5). As mentioned previously, when wings 32a and 32b are in an overlapped relationship while device 20 is in a collection configuration, the collective profile defined by wing sides 35a and 35b has a shape that generally conforms to the shape of the mons pubis and can provide alignment therewith, which in turn allows the user to reliably and consistently position device 20 on her anatomy to direct urine onto first and second side inner substrate surfaces 48a and 48b of substrate 21 during a urination event. As indicated previously, wing sides 35a and 35b conform to the perforations 25a and 25b.

In the testing configuration of FIGS. 2, wings 32a and 32b generally comprise a single peak and are respectively defined by edges 35a/37a and 35b/37b, all of which individually define monotonically increasing distances from longituidnal midline 38 along the width axis W when in the testing configuration and moving along the edges to 35a/37a and 36b/37b toward their respective wing peaks 33a/33b along the length axis L. The wing edges 37a/37b are proximal of their respective wing peaks 33a/33b and begin at first substrate end 26. The wing edges 35a/35b are distal of their respective wing peaks 33a/33b and terminate at approximately the horizontal midline 50 of substrate 21. Generally, the proximal wing edges 37a/37b have steeper slopes along the substrate length L axis than do their distal wing edges 35a/35b. Wing edges 37a/37b also have a point of inflection (where the curvature changes from concave up to concave down) approximately half-way along their lengths.

Referring again to FIG. 2, device 20 is in a testing configuration in which device 20 is preferably placed on a horizontal surface so that sample test strips 54a and 54b lay flat, which is desirable for lateral flow assembly strips. In FIG. 2, the first and second side inner substrate surfaces 48a and 48b of respective sides 22a and 22b are preferably coplanar. Sample test strips 54a and 54b may test for the same unknown or different ones. Sample test strip 54a has an exposed portion 56a and a covered portion 58a. Similarly, sample strip 54b has an exposed portion 56b and a covered portion 58b. The covered portions 58a and 58b contain the sample strip reagents are sealed and protected from contamination, such as due to urine overshoot, by covering layer 52. Covering layer 52 is preferably secured to side inner substrate surfaces 48a and 48b of substrate 21 snugly enough to retain sample test strips 54a and 54b within the space between covering layer 52 and side inner substrate surfaces 48a and 48b of substrate 21. In addition, exposed portions 56a and 56b preferably extend beyond covering layer 52 but not beyond second end 29 of substrate 21. Thus, sample strips 54a and 54b preferably lie fully within the perimeter of substrate 21, which reduces the likelihood of contamination of exposed portions 56a and 56b. In certain examples the distal most ends 55a and 55b of sample test strips 54a and 54b are at least about 0.15 inches, preferably at least about 0.12 inches, and more preferably, at least about 0.24 inches from second substrate end 29 along the length (L) axis. In the same or other examples, the lengths of exposed portions 56a and 56b of sample test strips 55a, 55b are from about 0.55 inches to about 0.75 inches, preferably from about 0.60 inches about 0.70 inches, and still more preferably from about 0.64 inches to about 0.68 The maximum exposed length of exposed sections 56a and 56b will typically be dictated by the sample test strip manufacturer. As a percentage of the length of substrate 21 from first end 26 to second end 29, the lengths of exposed portions 56a and 56b range from about seven (7) percent to about fifteen (15) percent, preferably from about 8 percent to about 14 percent, and more preferably from about ten (10) percent to about twelve (12) percent. As a percentage of the lengths of exposed portions 56a and 56b, the distances from distal sample test strip ends 55a, 55b to second substrate end 29 range from about 30 to about 45 percent, preferably from about 32 percent to about 42 percent, and more preferably from about 34 percent to about 40 percent.

The sample test strips 54a, 54b are preferably configured to maximize the test strip surface area contacted by urine under low flow conditions. Because longitudinal midline 38 will define the low point of the low path along substrate 21, it is desirable to position sample test strips 54a and 54b as close as possible to longitudinal midline 38 along width axis W without abutting the score line that defines longitudinal midline 38 so that the individual strips 54a, 54b do not bend in the sealed configuration of FIG. 1 or the collection configuration of FIG. 5. In certain examples, the width (W) axis spacing between sample test strips 54a and 54b is from about 0.06 inches to about 0.20 inches, preferably from about 0.08 inches to about 0.15 inches, and more preferably from about 0.09 inches to about 0.12 inches. The sample test strips 54a and 54b have lengths that are preferably parallel to the length of substrate 21 along length axis L and are preferably spaced apart symmetrically from longitudinal midline 38. This configuration helps maximize the contact of urine with exposed portions 55a, 55b of strips 54a, 54b even when the urine volume is low.

The material used for covering layer 52 preferably ensures that test strips 54a, 54b remain uncontaminated and easy to read while allowing for visual inspection and scanning of the test results. In certain preferred examples, covering layer 52 is transparent and/or translucent. Due to its transparency, chemical inertness, and waterproof properties, one suitable material for covering layer 52 is clear polypropylene (PP). Cellophane is another suitable material for covering layer 52. Cellophane is transparent, chemically inert to urine and waterproof. It is also biodegradable, providing an environmentally friendly alternative to other plastics.

Referring again to FIG. 2, the peak to peak distance between wing peak 33a and wing peak 33b along the width axis (W) relative to the length of substrate 21 along the length axis L (from first end 26 to second end 29) is preferably from about 0.6 to about 1.0, preferably from about 0.7 to about 0.8, and more preferably from about 0.8 to about 0.85. The distances from longitudinal midline 38 are half of these values. The distance between peaks 33a and 33b along the width axis W relative to the width of device 20 is preferably about 1.0 because when the device 20 is in the testing configuration of FIG. 2, the width of the device 20 is defined by the peak to peak distance between peaks 33a and 33b along the width axis.

Distally of the horizontal midline 50, when device 20 is in the testing configuration of FIG. 2, the shape of substrate 21 is generally rectangular and has a minimum width along the width axis W that is from about 60 percent to about 70 percent, preferably from about 62 percent to about 68 percent, and more preferably from about 64 percent to about 68 percent of the maximum width of substrate 21 along the width axis W (defined between the wing peaks 33a and 33b).

The distance from peaks 33a and 33b to first substrate end 26 along the length axis L is preferably from about 15 percent to about 25 percent, more preferably, from about 17 percent to about 22 percent, and still more preferably, from about 19 to about 21 percent of the length of device 20 from first substrate end 26 to second substrate end 29 along the length axis L.

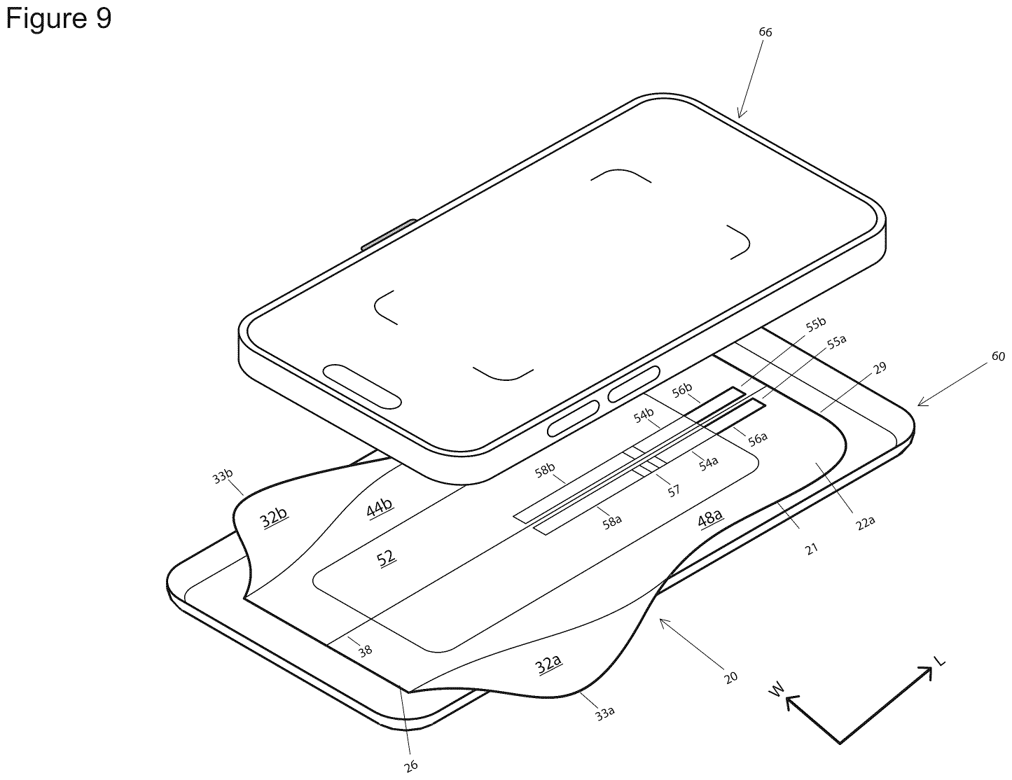

The transparent and/or translucent covering layer 52 allows the user to view the test results (e.g., result lines 57 in FIG. 9). The covering 52 may have an adhesive underside that adheres covering 52 to first and second side inner substrate surfaces 48a and 48b of substrate 21. Alternatively, a border region of the covering layer 52 may be adhered to the first and second side inner substrate surfaces 48a and/or 48b.

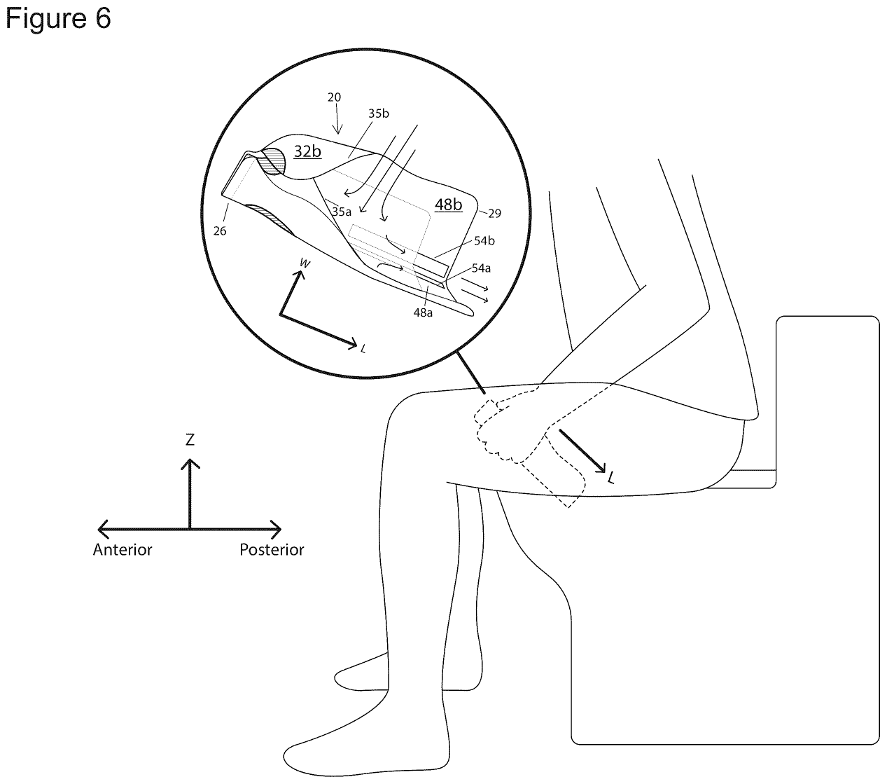

Referring to FIG. 5 device 20 is shown in a collection configuration. In the collection configuration, width-wise sealing section 40 has not been removed and holds portions of first end 26 proximate longitudinal midline 38 together. Thus, substrate sides 22a, 22b at first end 26 of substrate 21 cannot both lay flat in a coplanar configuration. The user holds the wings 32a and 32b in an overlapping relationship so that a portion of the first and second side inner substrate surfaces 48a and 48b of substrate 21 located between horizontal midline 50 and first end 26 is covered by wings 32a and 32b. Thus, the width-wise scaling section 40 and wings 32a and 32b define a closed end so that urine cannot exit device 20 at first substrate end 26. Because the substrate sides 22a and 22b are not joined between horizontal midline 50 and second substrate end 29, the first and second side inner substrate surfaces 48a and 48b are open and exposed between horizontal midline 50 and second substrate end 29, allowing urine to flow over sample strips 54a and 54b and exit the substrate 21 at second end 29. In FIG. 5, wing sides 35a and 35b cooperatively define an anatomical alignment ledge 37, as described previously. The user places her finger around finger engagement region 46 and thumb engagement portions 36a and 36b. The user can hold the device in a single hand as depicted in FIGS. 5 and 6 and align the anatomical alignment edge 37 with the mons pubis so that first substrate end 26 is vertically elevated above second end 29 and the length axis L of the device 20 is oriented at an angle that slopes anteriorly to posteriorly with respect to the woman's anatomy when moving downward along the vertical (z) axis.

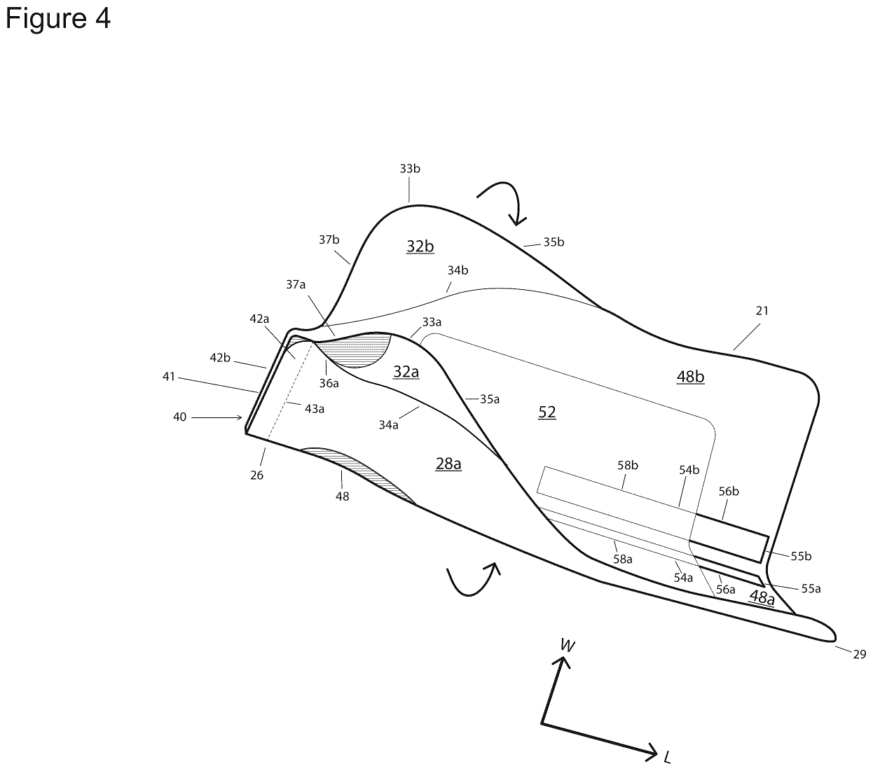

A method of using the urinalysis and collection device 20 will now be described. The device 20 is received in the sealed configuration shown in FIG. 1. In the sealed configuration, the sample testing strips 54a and 54b are concealed from view and, preferably, protected from the incursion of atmospheric moisture. The user then converts the device 20 from the sealed configuration to the collection configuration by removing longitudinal sealing section 23 as illustrated in FIG. 3. This is preferably done by initiating a tear at notches 44a and 44b (not visible in FIG. 1) and separating longitudinal sealing section 23 from substrate 21 along perforations 25a and 25b, each of which corresponds to a respective substrate side 22a and 22b. The perforations 25a and 25b follow the dashed lines and the wing contours 35a/36a and 35b/36b. The width-wise sealing section 40 remains in place. The sides 22a and 22b are then spaced apart from one another as shown in FIG. 4.

As shown in FIG. 5, wing 32b is overlapped on top of wing 32a. However, either wing 32a, 32b can overlap the other. While sitting on a toilet, the user preferably uses a single hand to hold thumb engagement region 36b of wing 32b over thumb engagement region 36a of wing 32a while wrapping the fingers around the outer surfaces 28a and 28b of substrate 21 so the fingers engage finger engagement section 46. Thus, in FIG. 5 device 20 is in the collection configuration and has the appearance of a closed-end scoop or a partial-funnel that is inverted. As indicated in FIG. 5, sample test strips 54a and 54b are already affixed in a location that is proximate longitudinal midline 38 and in a direction that is parallel to the length of device 20 and the direction of urine flow.

Referring to FIG. 6, the user then positions the anatomical alignment ledge 37 defined by wing sides 35a and 35b in alignment with the mons pubis, with the device 20 sloping from the anterior to posterior direction of her anatomy when moving vertically downward along the vertical (z) axis. In a preferred implementation, the user positions the anatomical alignment ledge 37 against the inferior part of the mons pubis, immediately superior to the clitoral hood. Owing to their shape and preferred paper board materials of construction, wings 32a, 32b may be gently pressed against the body.

This orientation fixes the relative location of the first and second side inner substrate surfaces 48a, 48b of substrate 21, the wings 32a, 32b and the user's urethral opening. This ability to readily and quickly align the device 20 with the female anatomy also advantageously allows the user to wait until a mid-stream portion of a urination event before collecting and testing urine. It has also been found that the relative positioning of wings 32a, 32b first and second side inner substrate surfaces 48a, 48b and sample strips 54a, 54b allows device 20 to be reliably used across a variety of female anatomies.

With the device 20 thusly positioned, the user urinates, onto the first and second side inner substrate surfaces 48a and 48b as shown by the downwardly-angled lines in the call-out of FIG. 6. The wings 32a and 32b effectively act as a splash guard when thusly located. As the call-out arrows indicate, owing to the geometry of the device 20 when positioned as described, urine will naturally flow vertically downward along the z axis and into the toilet, in a direction from the woman's anterior region to her posterior region. The longitudinal midline 38 defines the low point of a flow channel defined by the sides 22a and 22b of substrate 21. The alignment of the lengths of the sample test strips 54a and 54b is substantially parallel to the longitudinal midline 38, and the positioning of the test strips 55a and 54b helps to maximize the contact between the user's urine and test strips 54a, 54b. The shape of device 20 advantageously slopes downward when moving posteriorly from the anterior side of the user, allowing the first and second side inner substrate surfaces 48a and 48b distal of wings 32a and 32b to tuck over the vulvar anatomy.

Unlike many known devices, this shape also allows device 20 to be positioned like a “reverse funnel” because when it is aligned with the seated female anatomy, it utilizes space under the body, eliminating or minimizing the need for awkward squatting positions. In contrast, devices that ramp in the other direction will be more likely to abut the toilet seat and/or bowl. It has been found that the ability to perform such urine collection and testing when seated allows more women with varying anatomies to successfully test their urine on a standard toilet with minimal back splash. This alignment in turn avoids the need or the user to “aim” in order to ensure the urine contacts the sample test strips 54a, 54b.

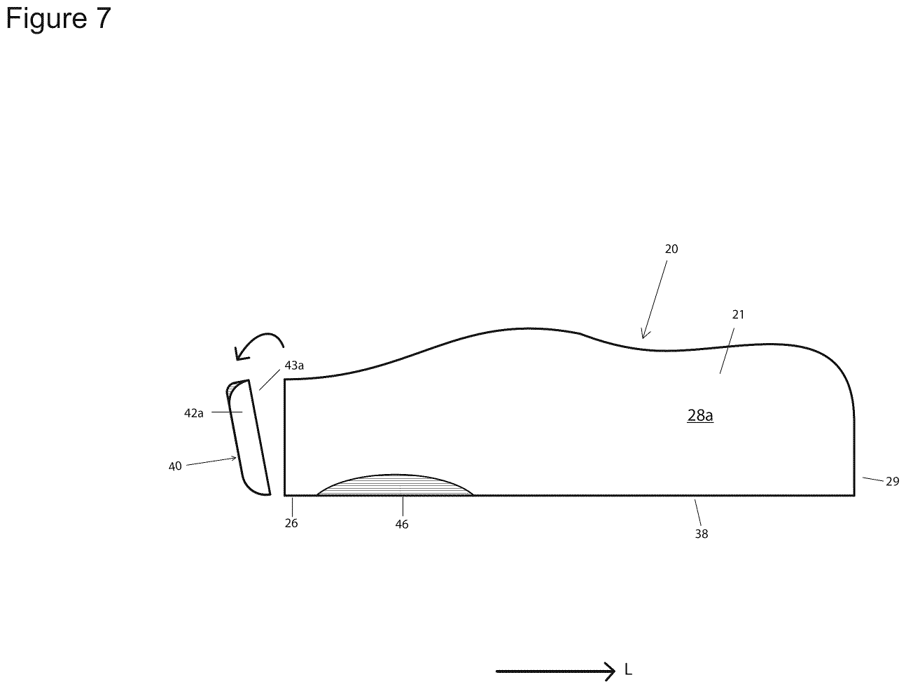

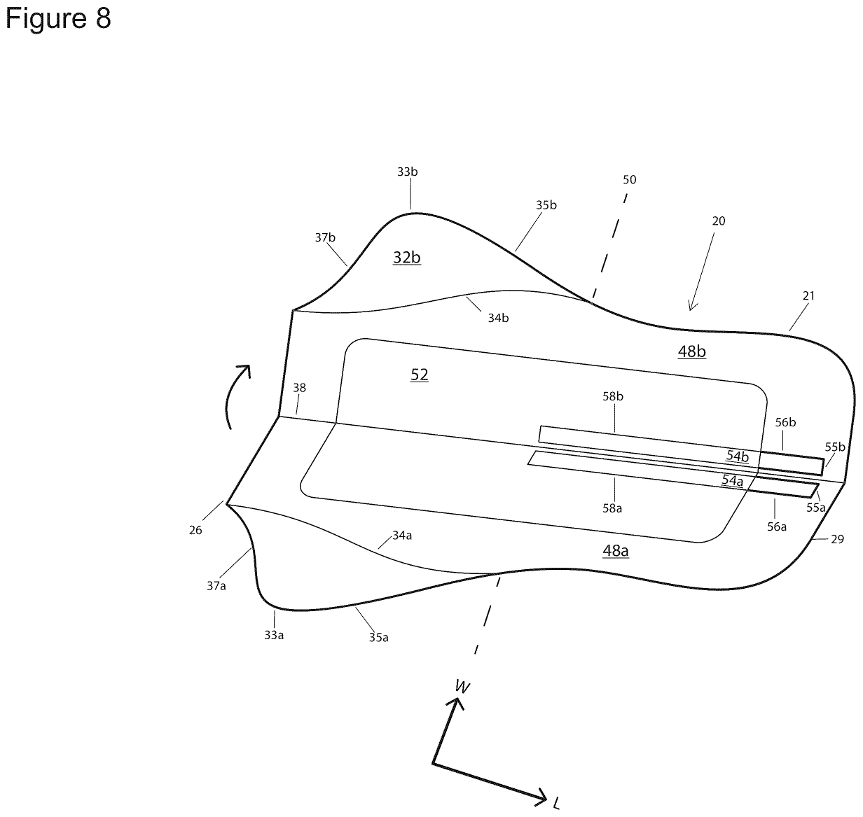

Referring to FIG. 7, to convert device 20 from the collection configuration to the testing configuration, width-wise sealing section 40 is removed by separating it along perforations 43a and 43b (not visible). As shown in FIG. 8, the removal of width-wise sealing section 40 allows the substrate sides 22a and 22b to be laid on a flat horizontal surface so that the first and second side inner substrate surfaces 48a and 48b are coplanar. This in turn causes the sample test strips 55a and 54b to be horizontal and coplanar and facing in the same direction, which is particularly beneficial for sample test strips that are lateral flow assays. Referring to FIG. 9, the covered portions 58a and 58b of sample test strips 54a and 54b can be viewed through transparent and/or translucent covering layer 52 so that results 57 can be seen with the naked eye. Once viewing is completed, device 20 can be discarded. The use of a transparent and/or translucent covering layer 52 also allows the covered portions 58a, 58b (which is where the reagent and urine react to perform the desired urinalysis), to be imaged for transmission to a remote server or third party for analysis. The planarity of the covering layer 52 also reduces imaging artifacts that can occur when light is incident on a non-planar film.

Some sample test strip suppliers have users scan their sample test strips and then process the scanned images to determine the test results. Referring to FIG. 9, device 20 is in a holding tray 60 that maintains the alignment and planarity of the substrate 21, apart from wings 32a, 32b. Smartphone 66 is positioned at an imaging distance to capture images of test strips 54a, 54b and transmit them to a local program on smartphone 66 or a remote server.

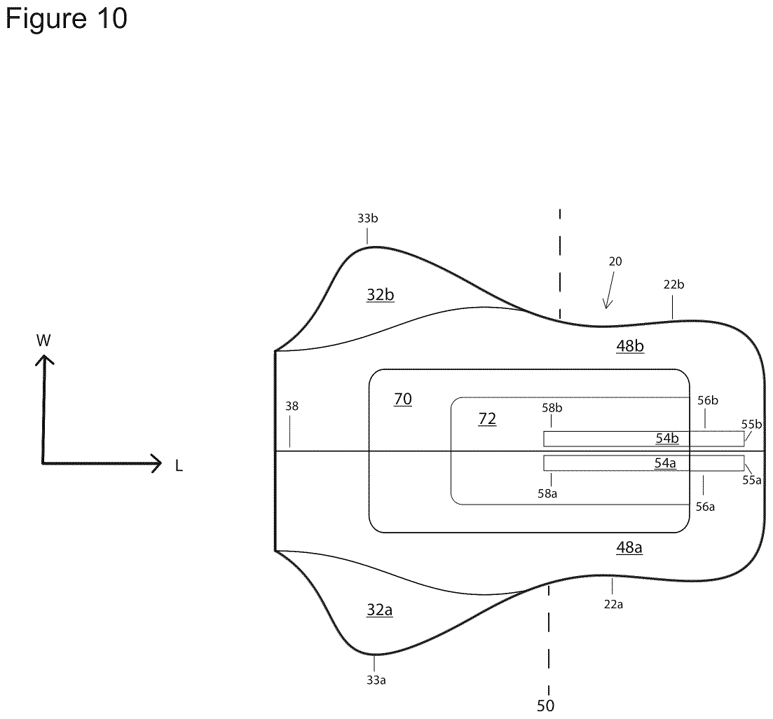

Referring to FIG. 10, a modified version of device 20 is shown. Like numerals refer to like components from the earlier figures. In this embodiment, a non-adhesive, transparent and/or translucent covering layer 72 lay immediately over the sample test strip 54a, 54b and a second adhesive layer 70 layer over the top of non-adhesive covering layer 72. The second adhesive covering layer 70 is larger in surface area than non-adhesive covering layer 72. Thus, the second adhesive covering layer 70 directly engages parts of the first and second side inner substrate surfaces 48a, 48b of substrate 21 and thereby holds the non-adhesive covering layer 72 firmly in place against the sample test strips 54a, 54b. This embodiment avoids contacting sample test strips 54a, 54b with adhesive.

A method of manufacturing urinalysis and collection device 20 will now be described. The manufacturing process is designed to be simple and cost-effective while ensuring high quality and consistency. In accordance with one method, substrate 21 can be die cut from a single sheet of paperboard into the shape shown in FIG. 2.

Covering layer 52 is separately cut from a sheet of the selected material (e.g., clear polypropylene or cellophane). It is preferably attached to first and second side inner substrate surfaces 48a and 48b to define a pocket that is not so tight that it might impede the laminar flow of urine from the exposed portions 56a, 56b of test strips 54a, 54b into the respective covered portions 58a, 58b of test strips 54a, 54b while also ensuring that urine does not seep beneath covering layer 52.

Beyond testing, the 20 device serves as a convenient tool for urine collection. This provides a hygienic and mess-free solution for sample acquisition, which can then be used for further laboratory analysis or diagnostic testing. Its user-friendly design makes it suitable for home use, allowing individuals to monitor their health conveniently and discreetly. The case of use and straightforward instructions ensure that anyone can perform necessary urine tests without professional assistance. It also facilitates self-testing when away from home, such as when camping, hiking, or traveling.

The device is adaptable for various applications, including midstream urine test strips, comprehensive urinalysis, and simple urine collection. Its versatility makes it suitable for clinical environments, home use, and outdoor activities.

What is claimed is:

1. A urinalysis and collection device having a sealed configuration, a collection configuration, and a testing configuration, the urinalysis and collection device comprising:
a substrate having an outer substrate surface and an inner substrate surface; and
a sample testing section comprising at least one sample test strip disposed on the inner substrate surface, wherein in the testing configuration, a first portion of the at least one sample test strip is covered, and a second portion of the sample test strip is uncovered, and in the sealed configuration, the substrate is folded and entirely encloses the at least one sample test strip.

2. The urinalysis and collection device of claim 1, further comprising a longitudinal removable sealing section, wherein the substrate comprises a first proximal end and a second distal end spaced apart along a length axis, a first substrate side adjacent a second substrate side along a width axis, and in the sealed configuration, the longitudinal removable sealing section is removably attached to each of the first substrate side and the second substrate side.

3. The urinalysis and collection device of claim 2, wherein the inner substrate surface comprises a first side inner substrate surface and a second side inner substrate surface, and in the sealed configuration, the first and second side inner substrate surfaces are in facing opposition to one another, and in the testing configuration, the first and second side inner substrate surfaces are coplanar with one another.

4. The urinalysis and collection device of claim 2, further comprising a width wise sealing section, wherein in the sealed configuration, the width wise sealing section is removably attached to respective portions of the first substrate side and the second substrate side at the first end of the substrate.

5. The urinalysis and collection device of claim 2, wherein the substrate includes a longitudinal midline and a horizontal midline, the first substrate side has a first wing that projects away from the longitudinal midline and the second substrate side has a second wing that projects away from the longitudinal midline, in the sealed configuration, the first and second wings project away from the longitudinal midline in the same direction, and in the testing configuration, the first and second wings project away from the longitudinal midline in opposite directions.

6. The urinalysis and collection device of claim 5, wherein each of the first wing and the second has a point of maximum projection away from the longitudinal midline along the width axis, and the respective points of maximum projection away from the midline are located between the first end of the substrate and the horizontal midline.

7. The urinalysis and collection device of claim 6, wherein the point of maximum projection of the first wing from the longitudinal midline defines a distance along the width axis, the distance from the first end of the substrate to the second end of the substrate defines a second distance along the length axis, and the ratio of the distance along the width axis to the distance along the length axis is at least 0.3.

8. The urinalysis and collection device of claim 1, wherein the at least one sample test strip is covered by a covering layer that is translucent and/or transparent.

9. The urinalysis and collection device of claim 1, wherein the at least one sample test strip is a laminar flow test strip.

10. The urinalysis and collection device of claim 9, wherein the laminar test strip is one selected from a hormone test strip, a pregnancy test strip, and a drug testing test strip.

11. The urinalysis and collection device of claim 1, wherein the substrate has a length along a length axis, the at least one sample test strip has a length, the length of the at least one sample test strip is parallel to the length axis.

12. The urinalysis and collection device of claim 1, wherein the substrate a comprises a first end and a second end spaced apart along a length axis, in the collection configuration, a portion of the inner substrate surface proximate the first end of the substrate is covered by the wings and a portion of the inner substrate surface proximate the second end of the substrate is open.

13. The urinalysis and collection device of claim 1, wherein the substrate comprises moisture resistant paperboard.

14. A method of collecting and testing female urine, comprising:
providing a urinalysis collection and testing device comprising a substrate having an inner substrate surface and an outer substrate surface, a first end spaced apart from a second end along a length axis, and first and second substrate sides adjacent one another along a width axis, wherein the first substrate side has a first wing, the second substrate side has a second wing parallel to the first wing;
folding the first and second wings toward one another to overlap the first and second wings, thereby covering a proximal portion of the inner substrate surface and defining an anatomical alignment ledge for aligning the device with an anatomical structure
directing urine onto a distal portion of the inner substrate surface between the anatomical alignment ledge and the second end of the substrate along the length axis.

15. The method of claim 14, wherein the step of directing urine onto the inner surface of the substrate between the anatomical alignment ledge and the second end of the substrate comprises holding the wings together with one hand and placing the anatomical alignment ledge in engagement with the mons pubis.

16. The method of claim 14, wherein the urinalysis collection and testing device comprises a sample testing strip partially covered by a covering layer that secures the testing strip to the inner surface of the substrate such that a portion of the sample testing strip extends beyond the covering layer and no portion of the sample testing strip extends beyond a boundary of the substrate.

17. The method of claim 16, further comprising the step of positioning the substrate such that the first end of the substrate is vertically higher than the second of the substrate to flow urine over the sample testing strip.

18. The method of claim 17, further comprising placing the urinalysis collection and testing device in a testing configuration by folding the first and second substrate sides away from one another.

19. The method of claim 14, wherein the step of providing a urinalysis collection and testing device comprises providing the urinalysis collection and testing device in a sealed condition in which the first and second substrate sides are connected by a removable longitudinal sealing section such that at least one sample sealing strip is fully enclosed within an interior region of the device, and the interior region of the device is defined by the inner substrate surface and an inner surface of the removable longitudinal sealing section, the method further comprising detaching the longitudinal sealing section from the first and second sides.

20. The method of claim 15, wherein the step of directing urine onto the inner substrate surface of the substrate between the anatomical alignment ledge and the second end of the substrate comprises placing the substrate proximate the mons pubis during a mid-stream portion of a urination event.
